(19) United States
(12) Patent Application Publication
(10) Pub. No.: US 2010/0049189 A1
(43) Pub. Date: Feb. 25, 2010

(54) DEVICE AND METHOD FOR TREATING ANNULAR ORGAN STRUCTURE

(76) Inventor: Duane Dickens, San Clemente, CA (US)

Correspondence Address: DALINA LAW GROUP, P.C., 7910 IVANHOE AVE. #325, LA JOLLA, CA, 92037, US

(21) Appl. No.: 12/177,780

(22) Filed: Jul. 22, 2008

Related U.S. Application Data
(60) Provisional application No. 60/951,226, filed on Jul. 22, 2007.

(51) Int. Cl. A61B 18/18

(52) U.S. Cl. 606/41

(57) ABSTRACT

System and method for repairing an annular organ structure such as heart valve including valve leaflet, chordae tendinae, papillary muscles and the like. Provides a deployable structure in the form of a plurality of tissue-contactor members with integrated tissue-shrinkable energy-emitting elements. Said plurality of tissue-contactor members in a deployed state having a configuration of radially expanded middle region suitable for contacting the inner wall of an annular organ structure for effectively applying tissue-shrinkable energy site-specifically. May be deployed into the heart using a minimally invasive surgical procedure. Reshaping collagen-rich tissue through the emission or generation of heat or radiation, modifying the tissue through the process of denaturation. Configuration of deployable structures at distal section of system enables method of treatment of an annular organ structure without an interruption of flow of blood or bodily fluid through the valvular annulus.

This application claims benefit of U.S. Provisional Patent Application Ser. No. 60/951,226 filed Jul. 22, 2007, the specification of which is hereby incorporated herein by reference.

### BACKGROUND OF THE INVENTION

1. Field of the Invention

Embodiments of the invention described herein pertain to the field of medical devices and methods for treating and/or repairing malfunctioning annular organ structure. More particularly, but not by way of limitation, one or more embodiments of the invention enable a catheter system that selectively contacts the tissue of an annular organ structure in order to tighten and stabilize the annular organ structure or adapted to repair the valvular annulus defect of a patient.

2. Description of the Related Art

Regurgitation (leakage) of the mitral valve or tricuspid valve can result from many different causes, such as an ischemic heart disease, myocardial infarction, acquired or inherited cardiomyopathy, congenital defect, myxomatous degeneration of valve tissue over time, traumatic injury, infectious disease, or various forms of heart disease. Primary-heart-muscle disease can cause valvular regurgitation through dilation, resulting in an expansion of the valvular annulus and leading to the malcoaptation of the valve leaflets through overstretching, degeneration, or rupture of the papillary-muscle apparatus, or through dysfunction or malpositioning of the papillary muscles. This regurgitation can cause heart irregularities, such as an irregular heart rhythm, and itself can cause inexorable deterioration in heart-muscle function. Such deterioration can be associated with functional impairment, congestive heart failure and significant pain, suffering, lessening of the quality of life, or even death.

Many repair techniques that address valvular disease at the annular level in hopes that the valvular annulus will coapt. Attempts to decrease annular size, transplant chordae, or resect portions of the valvular annulus hope to create an architecture in which the valvular annulus will once again coapt have met with a limited success. The variability of the subvalvular apparatus and the numerous pathologies of, for example, mitral valve regurgitation often complicate appropriate repair technique selection. Malcoaptation of the valvular annulus is only indirectly addressed by various repair techniques.

Therefore, there is a need to have a less surgically invasive catheter-based approach for repairing an annular organ structure by selectively contacting tissue and using tissue-shrinkable energy for reducing and/or shrinking a tissue mass.

### BRIEF SUMMARY OF THE INVENTION

One or more embodiments of the invention provide a system and method for repairing an annular organ structure of a heart valve, an annular organ structure of a venous valve, a valve leaflet, chordae tendinae, papillary muscles, a sphincter, and the like. In a minimally invasive surgical procedure, the system may be deployed into the heart via a catheter percutaneously or via a cannula through a percutaneous intercostal penetration.

In other embodiments, the system may be in a form of surgical hand-held apparatus during an open chest procedure. The system may be deployed into a sphincter via trans-thoracic or trans-abdominal approaches or via urogenital or gastrointestinal orifices. The system may be deployed into a venous valve using local surgical approaches or by percutaneous access into the venous system.

In the above embodiments, the invention provides a tissue-shrinkable energy that may be applied to the target annular organ sufficient to treat the target organ structure. The tissue-shrinkable energy may be cryogenic energy, radiofrequency energy, or high frequency current. In one or more embodiments of the invention, the system and method delivering a high frequency current may include but not limited to radiofrequency, focused ultrasound, infrared, or microwave energy, wherein the high frequent current is applied to the target organ structure.

Accordingly, one or more embodiments of the invention provide an apparatus for the application of tissue-shrinkable energy to an annular organ tissue site comprised of collagen. One or more embodiments of the invention provide a catheter-based minimally surgically invasive system that intimately contacts the tissue of an annulus in order to tighten and stabilize a substantial portion of the dysfunctional annular organ structure simultaneously or sequentially. The step of intimately contacting may be assisted by deployable structures at the distal segment of flexible guiding catheter of the device to position and stabilize the tissue-shrinkable energy-emitting elements relative to anatomic structures. The tissue-shrinkable energy elements may transmit an effective amount of the tissue shrinkable energy through a medium onto the target annulus in order to tighten and stabilize a substantial portion of the dysfunctional annular organ structure. The system may position a distal deployable structure at the commissures of an annular organ such as mitral heart valve.

One or more embodiments of the invention provide an apparatus with a plurality of radially-expandable flexible semi-rigid tissue-contactor members located at the distal tip section of a catheter shaft for positioning tissue-contactor members, each member having a pre-shaped structure with tissue-shrinkable energy-emitting element that are deployed about the valvular annulus. As such in a deployed state, the configuration of the plurality of tissue-contactor members enables a method of treatment of an annular organ structure without the interruption of flow of blood or bodily fluid through the valvular annulus.

One or more embodiments of the invention provide a method for reshaping collagen rich tissue through the emission or generation of heat or radiation within the tissue modifying the collagen through the process of denaturation. When tissue is subjected to, for example, elevated temperatures, the collagen bonds denature in a predictable manner causing immediate shrinkage of the collagen fibers resulting in tissue shrinkage. The method may include the monitoring and control of temperature at the tissue surface and within the tissue structure which is achieved through the use of suitable temperature sensors on or near the energy application site or integrated with energy-emitting element.

In one or more embodiments of the invention, a system and method for treating an affected portion of annular organ structure includes, but not limited to, inserting at least one mono-polar or bi-polar tip electrode at the distal end of the system into annular organ structure at least proximal to the affected portion, energizing the electrode to emit a tissue-shrinkable energy to heat the affected portion; and measuring a temperature of the affected portion, wherein the energizing of the electrode is associated with the measured temperature. In an embodiment, the electrode is no longer energized upon the measured temperature reaching a desired temperature. In an embodiment, the method further comprises transmitting a signal associated with the measured temperature to a processor, wherein the processor compares the measured temperature to a designated termination temperature. In an embodiment, power supplied to energize the electrode is altered based on the transmitted signal. The electrode may be inserted directly into the affected portion or inserted directly into healthy tissue to treat the affected portion in at least one of below the healthy tissue or adjacent to the healthy tissue. In an embodiment, the desired temperature is in the range of about 40° C. to about 75° C.

One or more embodiments of the invention to provide catheter system and methods for providing tissue-shrinking energy to, for example, the mitral valve annulus of a heart without an interruption of blood flow through the valve. In such embodiments, the apparatus is arranged in ways such that the blood flows through the valve being treated during the application of tissue-shrinkable energy.

### BRIEF DESCRIPTION OF THE DRAWINGS

The above and other aspects, features and advantages of the invention will be more apparent from the following more particular description thereof, presented in conjunction with the following drawings wherein:

FIG. 1 depicts an overall view of one embodiment of catheter system in a deployed state having a plurality of radially expandable flexible semi-rigid tissue-contactor members on the distal end, each tissue-contactor member fitted with energy-emitting element.

FIG. 2 illustrates a close-up view of the distal tip section of one embodiment of the invention comprising a retracted tissue contactor members in a non-deployed state.

FIG. 3 illustrates a close up view of the distal tip section of the embodiment described in FIG. 2 in a deployed state.

FIG. 4 illustrates a close up view of the distal tip section of one embodiment of a catheter system in a deployed state having a plurality of semi-rigid tissue-contactor members in a configuration of two radially expandable sections.

FIG. 5 illustrates a cut away view of tissue-contactor member with a tissue-shrinkable energy-emitting element.

FIG. 6 illustrates a close up view of the distal section in a deployed state of one embodiment of a catheter system having a plurality of tissue-contactor members and tip electrodes.

FIG. 7 illustrates a close up view of the distal tip section in a deployed state of one embodiment of a catheter system having a plurality of tissue-contactor members in a configuration of two radially expandable sections and tip electrodes.

### DETAILED DESCRIPTION

A device and methods for treating annular organ structure will now be described. In the following exemplary description numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

In general, system comprises a catheter or similar probe having a plurality of flexible semi-rigid tissue-contactor members that self-expands radially for contacting about the commissures of an annular organ structure. Each tissue-contactor member may have at least one tissue-shrinkable energy-emitting element which is able to be turned on and off, and/or modulated between high and low intensities. The energy-emitting element is connected to an energy source or generator that can be controlled to treat tissue or cause the tissue tightening and/or shrinkage.

One or more embodiments of the invention provide a system having one or a plurality of energy-emitting elements. The energy-emitting element may be mounted on a substrate in a defined spatial pattern, or array. As used herein, the term “array” refers to an arrangement of energy-emitting elements. The array may be a regular array, such as a line, a series of column and rows, or a spiral, or a random array.

As used herein, the term “tissue-shrinkable energy” is intended to describe any energy that cause geometrical modification of tissue or cause tissue shrink of its original dimension. The tissue-shrinkable energy may also be useful as a stimulant for inducing a biologic repair process in a way that treated tissue becomes more resilient. The tissue-shrinkable energy is in term provided b energy-emitting element. In some embodiment, the tissue shrinkable energy is infrared energy, ultrasound energy, radiofrequency energy, microwave energy, electromagnetic energy, laser energy, or the like.

When a tissue-shrinkable energy such as moderate thermal energy, not ablation, is applied to, for example, a collagen molecule, the hydrogen bonds that hold the collagen molecule together are disrupted but the strong intermolecular bonds remain intact. The collagen collapses into random coils and the tissue shrinks or tightens during this energy-induced modification of the collagen. The result is a partial recovery or recreation of the mechanical properties of collagenous tissue. Therefore, one or more embodiments of the invention treat an annular organ structure by shrinking/tightening techniques through the application of tissue-shrinkable energy.

For monitoring local tissue temperature fluctuation, one or a plurality of temperature sensors may be adapted, coupled or integrated with one or more energy-emitting elements, or alternatively adapted or coupled to the tissue-contactor member. The temperature sensor allows for monitoring of local tissue temperature to guard against over heating of tissue. A temperature sensor may incorporate one or more temperature-sensing elements such as, for example, semiconductor-based sensors, thermisters, thermocouples, or fiber optic temperature sensors. Miniature temperature sensors known to one of ordinary skill in the art and suitable for this particular in vivo application may also be used.

An independent temperature monitor may be connected to the temperature sensor. A closed-loop control mechanism, such as a feedback controller with a microprocessor or a temperature controller, may be implemented for controlling the delivery of tissue-shrinkable energy to the target tissue based on temperature measured by the temperature sensor. Alternatively, an energy source with an integrated temperature monitoring circuit may be used to control the tissue-shrinkable energy power output supplied to the energy-emitting element.

The amount of tissue shrinkable energy as emitted by energy-emitting element is the amount effective to tighten and shrink tissue and not to cause any adverse impact on the mechanical properties of collagenous tissue. The temperature to tighten and shrink, for example, a collagenous tissue within a range of about 40° C. to 75° C. for a short period of time. The heat labile cross-linkages of collagen may be broken by thermal effects, thus causing the helical structure of the molecule to be destroyed (or denatured) with the peptide chains separating into individually randomly coiled structures of significantly lesser length. The thermal cleaving of such cross-links may result in contraction or shrinkage of the collagen molecule along its longitudinal axis by as much as one-third of its original dimension. It is such shrinkage of collagenous tissue that may effect a partial recovery or recreation of the mechanical properties of collagenous tissue.

Collagen shrinks within a specific temperature range, (e.g., 50° C. to 70° C. depending on collagen type), which range has been variously defined as: the temperature at which a helical structure collagen molecule is denatured; the temperature at which ½ of the helical superstructure is lost; or the temperature at which the collagen shrinkage is greatest. In fact, the concept of a single collagen shrinkage temperature is less than meaningful, because shrinkage or denaturation of collagen depends not only on an actual peak temperature, also on a temperature increase profile (increase in temperature at a particular rate and maintenance at a particular temperature over a period of time). For example, collagen shrinkage can be attained through high-energy exposure (energy density) for a very short period of time to attain “instantaneous” collagen shrinkage (e.g., 1-2 seconds). Longer time intervals between deliveries of tissue shrinkable energy allow slower rate of collagen shrinkage, affording the surgeon sufficient time to evaluate the extent of tissue shrinkage and to terminate tissue shrinkable energy delivery based on observation. Using the apparatus and methods of the present invention, the surgeon simply may terminate the power to energy-emitting element at any time during tissue shrinkage to gauge the correct amount of shrinkage.

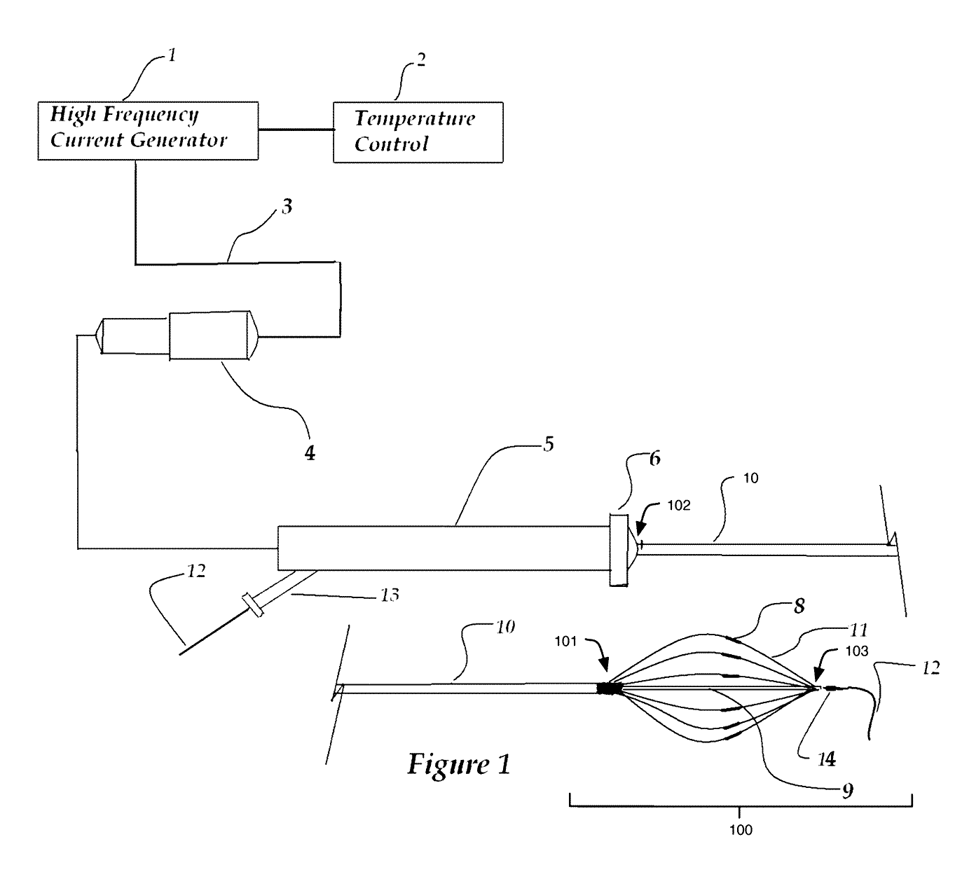

FIG. 1 illustrates an overall view of one embodiment of a catheter-based treatment system having a plurality of flexible semi-rigid tissue-contactor members 11, each having energy-emitting element 8 being integral of the tissue-contactor member 11, constructed in accordance with the principles of the invention comprises a flexible catheter shaft 10 having a distal tip section 100, a distal end 101, a proximal end 102, and at least one lumen extending therebetween. As shown in FIG. 1, elongated catheter shaft 10 preferably is sufficiently flexible to bend about its longitudinal axis combined with a high degree of longitudinal stiffness (resistance to shortening) and torqueability. In addition, elongated shaft 10 may comprise a resilient material capable of being springably formed to either a repose curved or linear configuration.

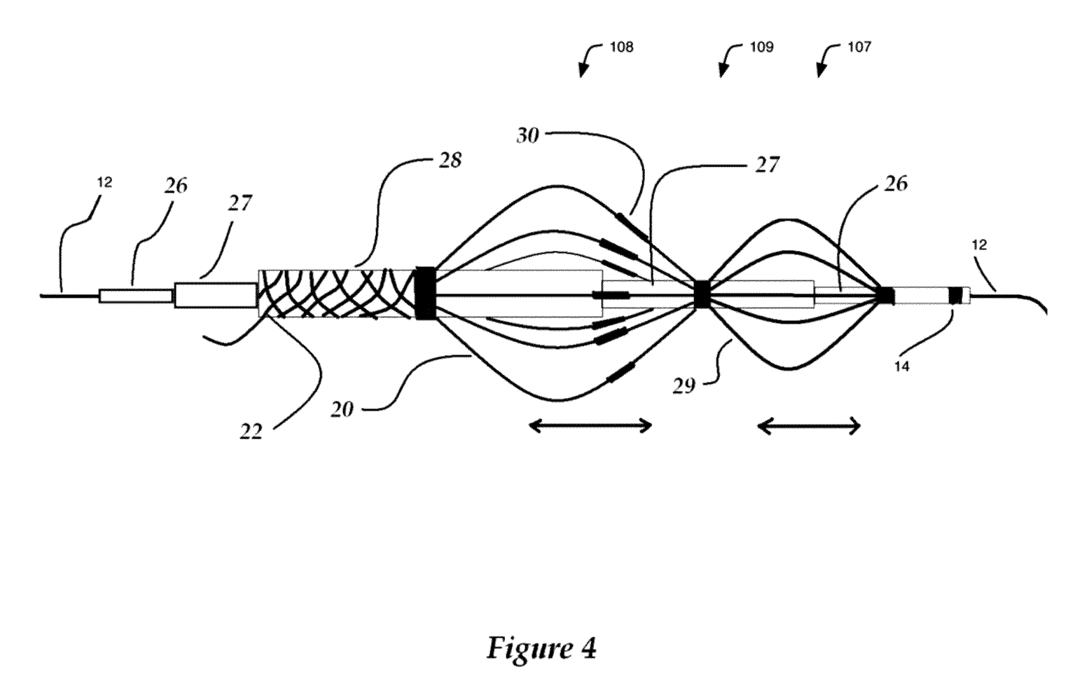

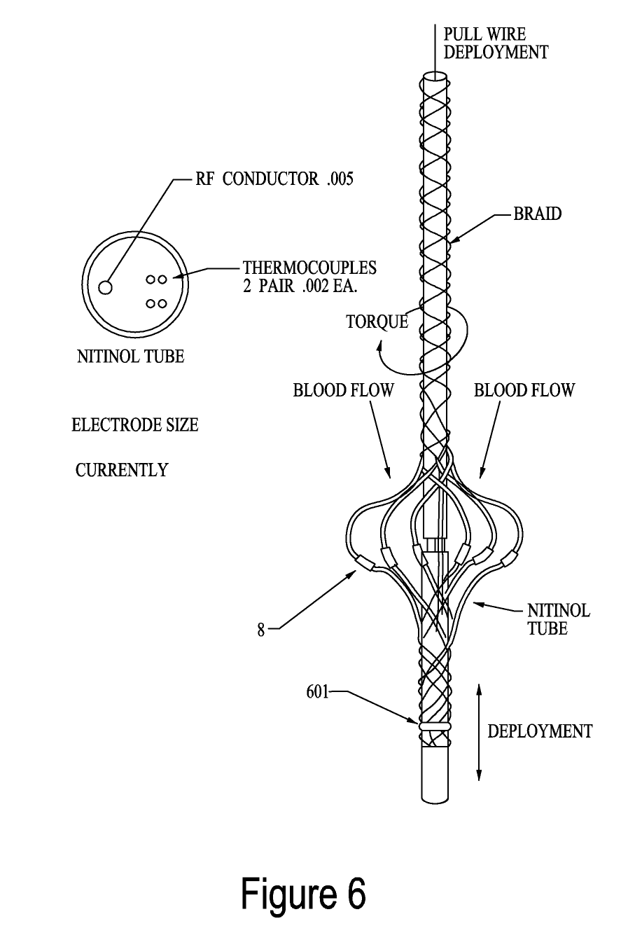

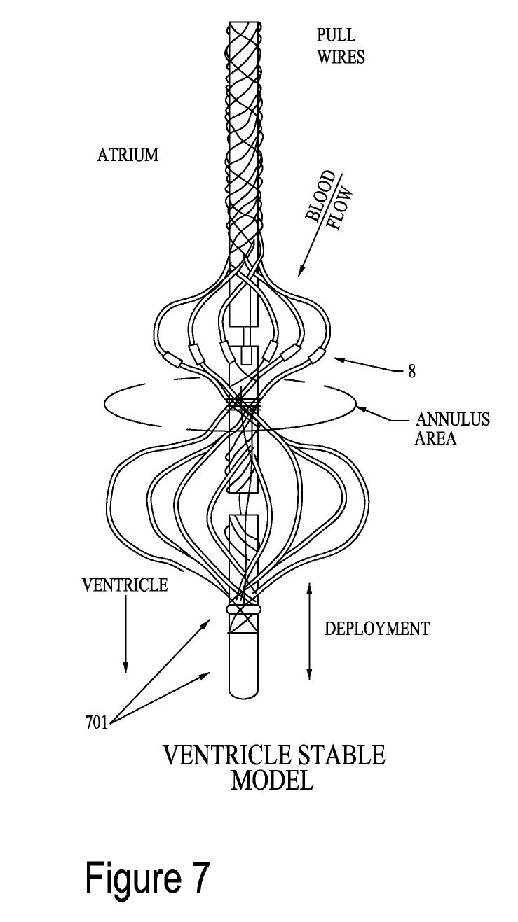

In one embodiment, the catheter system comprises an inner tubular member 9 extending from inside the at least one lumen of the catheter shaft 10 and a plurality of tissue-contactor members 11 located at the distal tip section 100 for contacting an inner wall of an annular organ structure when deployed. The tissue-contactor members may have certain variations (as shown in FIG. 4, FIG. 6 and FIG. 7) sharing the common characteristic of a configuration of radially expanded middle region suitable for contacting the inner wall of the annular organ structure for effectively applying tissue-shrinkable energy site-specifically.

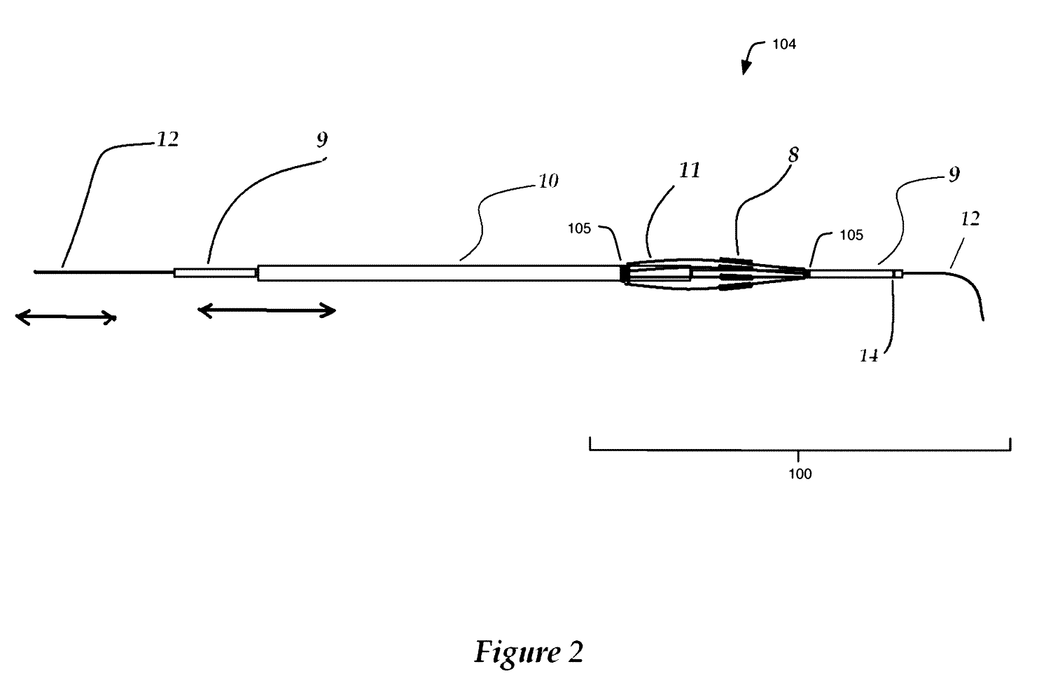

The plurality of tissue contactor-members 11 are deployable by extending the inner tubular member 9 out of the at least one lumen of the catheter shaft by an inner tubular member deployment mechanism 6 located at a handle 5. The distal end 103 of the tissue-contactor member 11 is coupled to the inner tubular member 9 capable of extending out and in therefrom the at least one lumen of the catheter shaft 10. The inner tubular member 9 optionally is coupled with a pull wire 12 to assist with reverse-deployment of the plurality of tissue contactor members 11 for a radially contracted configuration (as shown in FIG. 2). The tissue-contactor members 11 are preformed or expandable to have an appropriate shape configured to fit with the inner wall of the annular organ structure.

The handle 5 is attached to the proximal end 102 of the catheter shaft 10. The handle 5 comprises the inner tubular member deployment mechanism 13 and steering mechanism. The steering mechanism is to rotate and/or deflect the distal tip section of the catheter shaft for catheter maneuvering and positioning. In another embodiment, the steering mechanism at the handle comprises means for providing a plurality of deflectable curves on the distal tip section of the catheter shaft.

A connector 4, secured at the proximal end of the catheter system, is part of the handle section. The catheter system also comprises an energy source 1 such as a high frequency current generator, wherein an electrical conductor means for transmitting tissue-shrinkable energy (e.g., high frequency current) to energy-emitting elements (8 in FIG. 1, 8 in FIG. 2, 8 in FIG. 6 and 8 in FIG. 7) or tip electrodes (601 in FIG. 6 and 701 in FIG. 7) is provided. An embodiment provides tissue-shrinkable energy in a form of high frequency heat to collagen of tissue to a temperature range of about 40° C. to 75° C. or higher for at least a few second to cause collage shrink a fraction of its original dimension. The energy required from the high frequency current generator is generally less than 100 watts, typically less than 30 watts. In an embodiment, the high frequency current generator has a single channel and delivers the power to the energy-emitting elements and/or tip electrode continuously. In an embodiment, the high frequency energy emitted at the energy-emitting elements and/or tip electrode may be multiplexed by applying the energy in different waveform patterns (e.g., sinusoidal wave, sawtooth wave, square wave) over time as appropriate. In an embodiment, the affected tissue is continuously heated by the energy emitting elements and/or tip electrode for a desired amount of time. It should be noted that other power levels, desired temperatures, desired time periods, and/or energy patterns are contemplated based on type of affected tissue, materials used in the catheter system, frequencies and other factors.

FIG. 2 illustrates a close up view of the distal tip section 100 of the catheter system having a plurality of tubular tissue-contactor members 11 in a radially contracted configuration 104 in a non-deployed state. Each distal end of the tissue-contactor member may be coupled to an inner tubular member 9 by, for example, a retaining ring 105. Each of the tissue-contractor members 11 has an energy-emitting element 8 in a proximal position to the distal end. The inner tubular member 9 is in an extended configuration from the at least one lumen of the catheter shaft 10. The distal end of the inner tubular member 10 may be surrounded by a radioplague marker band 14 to aid fluoroscopic observation during manipulation of the catheter inside the body of a patient (e.g. vasculature). A pull-wire 12 is attached at the distal end of the inner tubular member. In this embodiment, the surgeon may use the pull wire 12 to pull the inner tubular member 9 into an extended configuration thus placing the plurality of tissue-contactor members 11 in a radially contracted configuration 104.

Tissue-contactor member embodiments are typically formed from a shape-memory material, such as Nitinol, having superelastic properities generally made from ratios of nickel and titanium. It is other shape memory materials for the tissue-contactor members are contemplated without digressing from the inventive concepts described herein such as shape memory plastics, polymers, and thermoplastics. The tissue-contactor members can be formed from a number of materials. For example, the tissue-contactor member can be formed from a biocompatible metal, metal alloy, polymeric material, or combination thereof. Examples of suitable materials include, but not limited to, medical grade stainless steel (e.g., 316L), titanium, tantalum, platinum alloys, nobium alloys, cobalt alloys, alginate, or combinations thereof. Other materials are also possible.

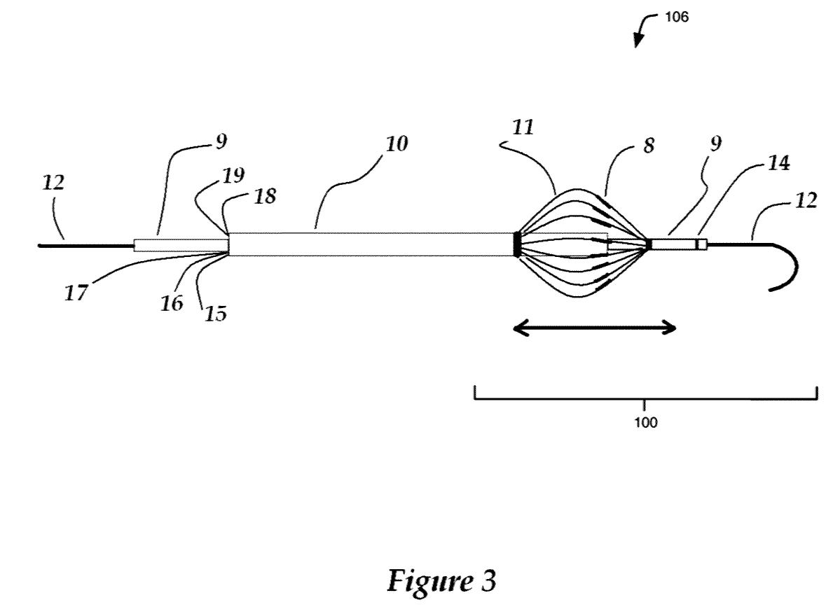

FIG. 3 illustrates a close up view of the distal tip section 100 of the embodiment described in FIG. 2 with the plurality of tissue-contactor members in a deployed state. The plurality of tubular tissue-contactor members is in a radially expanded configuration 106 having each distal end of the tissue-contactor member coupled to an inner tubular member in a retracted configuration from the at least one lumen of the catheter shaft.

FIG. 4 illustrates a close up view of the distal tip section of one embodiment of a catheter system in a deployed state having two sections of a plurality of semi-rigid tissue-contactor members 20 in a “double mound” configuration of radially expandable first section 107 and second section 108 and a narrow middle region 109 between the first section 107 and second section 108. The first section has energy-emitting elements 30 integrated with the plurality of tissue-contactor members 20. It be noted that both or either sections of the plurality of tissue-contactor can have integrated energy-emitting elements. A first inner tubular member is in a retracted configuration, extending from a lumen of a second tubular member, wherein the second tubular member is also in a retracted configuration, extending from at least one lumen of the catheter shaft. The first tubular member 26 and second tubular member 27 can assume a different retracted and/or extended configuration independently from each other thus the first section and second section of the plurality of tissue-contactor members can also assume radially expanded or contracted configuration independently of each other.

By having a “double mound” configuration, the structure provided by the two sections of the plurality of tissue-contactor members can exert adequate pressure to the surrounding tissue of an annular organ structure for stabilizing the placement of the distal section of the catheter system and/or suitable for compressively sandwiching the inner wall of the annular organ structure. The basic principle for the tissue-contactor member (as illustrated in FIG. 4 and FIG. 7) of one or more embodiments of the invention is to compress the target tissue (e.g., annulus, sphincter, tumor and the like) for enhanced site-specific application of tissue-shrinkable energy. The compression may come from sandwich-type setup, such as from two opposite tissue-contactor members at a suitable angle arrangement to compress the target tissue.

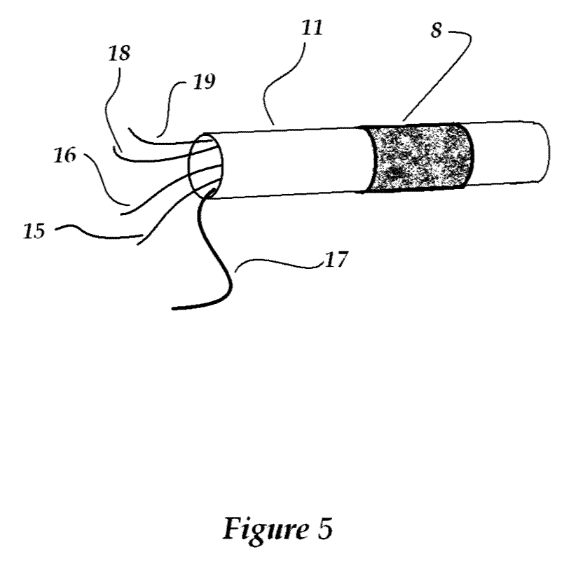

FIG. 5 illustrates a cut away view of a tubular tissue-contactor member 11 integrated with a tissue-shrinkable energy-emitting element 8. The energy-emitting element 8 is fitted with at least one temperature sensor (e.g., a thermocouple type, a thermister type, or any miniature temperature sensor known to one of ordinary skills in the relevant art) which are coupled via temperature sensing wires 15, 16, 17, 19 with the temperature controller 2 of FIG. 1. The energy-emitting element 8 is coupled to an electrical conductor means 17 for connecting the energy-emitting element to an energy source 1 of FIG. 1. The temperature sensing wires from the at least one temperature sensor are connected to one of the contact pins of the connector and externally connected to a transducer and to a temperature controller. The temperature reading is thereafter relayed to a closed-loop control mechanism to adjust the tissue-shrinkable energy output (e.g. high frequency energy) of the energy-emitting element. For example, the high frequency energy delivered by the energy-emitting element is thus controlled by the temperature sensor reading or by a pre-programmed control algorithm.

FIG. 6 illustrates a close up view of the distal section in a deployed state of one embodiment of a catheter system having a catheter shaft with at least one lumen, a plurality of flexible semi-rigid tissue-contactor members and bi-polar tip electrodes coupled at the distal end of a sliding distal tubular member pulled in towards the catheter shaft in a retracted position by a pull wire inside at least one lumen of the catheter shaft. The catheter system comprises a woven or braided plurality of tissue-contactor members attached on the surface of the catheter shaft and distal tubular member. The distal tubular member includes integrated bi-polar electrodes at the distal tip. The distal tubular member can be rotated around the longitudinal axis and in respect to the catheter shaft. In an embodiment, the distal tubular member is coupled to an inner tubular member which is coupled through at least one lumen of the catheter shaft to an inner tubular member deployment mechanism located at a handle. The rotation of the distal tubular member is carried out by, for example, rotating the inner tubular member deployment mechanism. The tissue-contactor members can be torqued such in a way in which a proximate section of the tissue-contactor members assumes a radially expanded configuration to have an appropriate shape configured to fit with the inner wall of the annular organ structure.

FIG. 7 illustrates a close up view of the distal section in a deployed state of one embodiment of a catheter system having a plurality of flexible semi-rigid tissue-contactor members having two sections, both in a radially expanded configuration, and bi-polar electrodes at the distal tip of a first distal tubular member. The first distal tubular member and a second distal tubular member are pulled toward the cathether shaft, each distal tubular member being pulled by pull wire. By bringing distal tubular members closer to the catheter shaft, the sections of the plurality of tissue-contactor members assume the radially expanded configuration. The first distal tubular member has bi-polar tip electrodes coupled at the distal end. During deployment of an embodiment, the annular area of an annular organ structure surrounds a narrow region between “double mound” structures of deployed sections of the plurality of tissue-contactor members. By having a “double mound” configuration, the structure provided by the two sections of the plurality of tissue-contactor members can exert adequate pressure to the surrounding tissue of an annular organ structure for stabilizing the placement of the distal section of the catheter system and/or suitable for compressively sandwiching the inner wall of the annular organ structure for site specific application of tissue-shrinkable energy.

The distal tip electrodes (601 in FIG. 6 and 702 in FIG. 7) are electrically connected to the energy source (e.g. RF generating device) that is located outside the patient's body. In an embodiment, the distal tip electrode emits the RF signals whereas the proximal tip electrode receive the signal to effectively spread the tissue-shrinkable energy in the form of RF signal through the tissue between the distal tip electrode and proximal tip electrode. In an embodiment, the proximal tip electrode is grounded. Alternatively, the proximal tip electrode has an opposite polarity to that of the distal tip electrode. It should be contemplated, however, that any of the embodiment described herein may utilize either the mono-polar or bi-polar configuration.

While the invention herein disclosed has been described by means of specific embodiments and applications thereof, numerous modifications and variations could be made thereto by those skilled in the art without departing from the scope of the invention set forth in the claims.

What is claimed is:

1. A device configured for shrinking a tissue of an annular organ structure of the body, wherein said device comprising:
An elongated catheter shaft defined with at least one lumen having a distal segment including a plurality of tubular tissue-connector members with an integrated energy-emitting elements and a proximal end attached to a handle and means for connecting to an energy source;
at least one temperature sensor at an energy-emitting element means adapted for measuring temperature of a inner wall of said annular organ structure;
said tissue contacting member having at least one energy-emitting element electrically coupled to said energy source; and,
at least one tubular member slidably disposed within said distal section for deploying said plurality of tissue-contactor members in a first configuration or a second configuration, wherein said second configuration is said plurality of tissue-contactor member are appropriately shaped to be positioned within or about annular structures within the body.
